(19) United States
(12) Patent Application Publication
(10) Pub. No.: US 2016/0333302 A1
(43) Pub. Date: Nov. 17, 2016

(54) ELECTROPORATION DEVICE FOR TRANSFERRING MATERIAL INTO CELLS, ELECTROPORATION APPARATUS COMPRISING SAME, AND ELECTROPORATION METHOD

(71) Applicant: PUKYONG NATIONAL UNIVERSITY INDUSTRY-UNIVERSITY COOPERATION FOUNDATION, Busan (KR)

(72) Inventors: Do Jin IM, Busan (KR); Byeong Sun YOO, Cheongju-si (KR); Su Nam JEONG, Namhae-gun (KR); Dong Pyo KIM, Pohang-si (KR); In Seok KANG, Pohang-si (KR)

(21) Appl. No.: 15/217,694

(22) Filed: Jul. 22, 2016

Related U.S. Application Data
(63) Continuation of application No. PCT/KR2014/011813, filed on Dec. 4, 2014.

(30) Foreign Application Priority Data
Jan. 23, 2014 (KR) 10-2014-0008404

(51) Int. Cl. C12M 1/42; C12N 15/82

(52) U.S. Cl. CPC C12M 35/02; C12N 15/8206

(57) ABSTRACT

The present invention relates to an electroporation device comprising: a droplet driving electrode which is in contact with and charges a first droplet containing cells and a second droplet containing a material to be delivered into cells and combines the first droplet and the second droplet, thereby generating a mixed droplet; and an electroporation electrode which applies voltage to the mixed droplet so as to perform electroporation inside the mixed droplet; to an electroporation apparatus comprising the electroporation device; to an electroporation method using the device; and to a method for transferring a material into cells.

### FIELD OF INVENTION

The present invention relates to an electroporation device for delivering a material into cells, an electroporation apparatus comprising the same, and an electroporation method. In particular, the present invention relates to an electroporation device for electrically charging droplets containing cells and droplets containing a material to be delivered into the cells, combining both droplets and controlling the combined droplets by electrophoresis, and performing electroporation; to an electroporation apparatus comprising the electroporation device; and an electroporation method using the device.

### BACKGROUND

A technic for delivering a genetic material to a cell so as to be expressed a desired character is a core technology in genetic engineering. A method for transferring a material to cells is classified into two categories depending on whether a virus is used or not. A method of using a virus is advantageous in that a material can be effectively delivered. However, a virus may cause a risk and an undesirable side effect, and it is difficult to design a suitable virus for individual cells. An example of a method without using a virus is an electroporation which temporally increases permeability of cell membranes by applying a high voltage electrical pulse in a moment, so as to make a material to be delivered into the cells. Among the methods without using a virus, the electroporation has been widely used because it can be applied regardless of the types of cells, and shows relatively high delivering efficiency. However, a commercialized electroporation apparatus has a limitation in that cell survival ratio is very low because it uses high voltage.

The commercialized electroporation apparatus has another limitation that it may cause cell contamination during electroporation, it requires an expensive power supply for maintaining electric current of several amperes at high voltage, and its consumables are also expensive. In addition, the commercialized electroporation apparatus is disadvantageous in that the deviation of experimental outcomes is relatively high due to its big size.

As an alternative of overcoming such limitations of the commercialized electroporation apparatus, an electroporation apparatus using a microfluidic technology has been developed. An electroporation apparatus using a microfluidic technology is advantageous in that because its size is small, it is possible to result in higher cell survival ratio with equivalent electroporation efficiency at a low voltage, compared with the commercialized electroporation apparatus, and it also requires small amount of materials. However, the prior art electroporation apparatus using a microfluidic technology has limitations on that cells which can be obtained at once are very small because it uses tiny amount of cells, and it is not user-friendly because it consists of micro elements.

Korean Laid-Open Patent Publication No. 2009-0018469 discloses an apparatus for evaluating cell electroporation efficiency of a method which uses a microfluidic device and a method for evaluating cell electroporation efficiency using the same. However, the apparatus disclosed in this application is not user-friendly because electroporation of cells is carried out inside micro-channels formed on a substrate. In addition, since the fluid to be introduced into micro-channels on the microfluidic device can contain extremely small amount of cells, such apparatus is not applicable to biological engineering that cells containing a material delivered thereto should be used after cultivation.

Therefore, there is a need to develop a novel electroporation device which can overcome limitations of both the above-mentioned commercialized electroporation apparatus and the apparatus adopting microfluidic technology, as well as can take advantages thereof.

### DESCRIPTION OF INVENTION

### SUMMARY OF INVENTION

In order to overcome the limitations of the prior art, the present invention provides an electroporation device which can be used for efficiently delivering a material into cells, an electroporation apparatus comprising the same, and a method for electroporation.

The present invention also provides an electroporation device which can directly charge a droplet containing cells and a droplet containing materials to be delivered to the cells, and combine the droplets by electrophoresis, so as to make it possible to perform electroporation inside the droplets, by which high efficiency of material delivery into the cells can be achieved with high cell survival ratio; an electroporation comprising the same, and a method of electroporation using the same.

The object of the present invention does not limit the above-mentioned, and any objects which are not explicitly mentioned can be clearly understood by a person of ordinary skill in the art in view of the description below.

### Technical Solution

The object of the present invention can be achieved by the technical solutions below:

1. An electroporation device comprising: a droplet driving electrode for forming a mixed droplet comprising a first droplet containing cells and a second droplet containing a material to be delivered into the cells; and an electroporation electrode for performing electroporation on cells in the mixed droplet.

2. An electroporation apparatus comprising: an electroporation device comprising, a droplet driving electrode for forming a mixed droplet comprising a first droplet containing cells and a second droplet containing a material to be delivered into the cells, and an electroporation electrode for performing electroporation on cells in the mixed droplet; a first power supply for applying voltage to the droplet driving electrode; and a second power supply for applying voltage to the electroporation electrode.

3. A method for electroporation comprising: introducing into an electroporation device a first droplet containing cells and a second droplet containing a material to be delivered into the cells; combining the first droplet and the second droplet so as to generate a mixed droplet; and performing an electroporation to the cells in the mixed droplet.

4. A method for electroporation comprising: introducing a droplet containing cells and a material to be delivered into the cells into the electroporation device according to any of claims 1 to 13 of the invention; and performing an electroporation on cells inside the droplet.

5. A method for delivering a material into cells comprising, performing electroporation of a droplet containing cells and a second droplet containing a material to be delivered into the cells using an electroporation device according to any of claims 1 to 13 of the invention, so as to deliver the material into the cells.

### Effect of Invention

The effects achieved by the present invention are as follows:

1. Higher material delivering efficiency and cell survival ratio are achieved;

2. An electroporation can be performed by the present invention with lower voltage and current compared with both commercialized and prior art electroporation apparatus, so as to let the cost for making an electroporation apparatus down;

3. It is possible to deal with millions of cells, equivalent to those which can be dealt with the commercialized electroporation apparatus, at once with higher material delivering efficiency and cell survival ratio;

4. Cells and a material in cells are dealt with in oil which is filled in an electroporation device, and thus, the cells do not contact with the surface of the device. Therefore, cell contamination can be minimized;

5. Since structure and the way of working of the electroporation device are simple, automation can be easily achieved through integration of components, by which consistency of results of electroporation can be achieved;

6. Structure of the device is simple, and therefore, even a person who is not familiar with the device can perform electroporation;

The effect of the present invention does not limit to those as mentioned above, and any effects which are not explicitly mentioned can be clearly understood by a person of ordinary skill in the art in view of any descriptions below.

### DESCRIPTION OF DRAWINGS

FIG. 1 shows main components of the electroporation apparatus of an embodiment of the present invention;

FIG. 2 shows side cross-section view of the process of performing electroporation in an electroporation device of an embodiment of the present invention;

FIG. 3 shows a process of performing electroporation and delivering a material into cells in the electroporation device in which its components were integrated and automated according to an embodiment of the present invention;

FIG. 4 shows an experimental process for demonstrating the process of a droplet driving, droplet combination and electroporation using an electroporation apparatus of an embodiment of the present invention;

FIG. 5 depicts an example of applied voltage profile for electroporation according to the present invention;

FIG. 6 depicts distribution of current which flowed through droplets by the voltage profile depicted in FIG. 5;

FIG. 7 shows that a genetic material was successfully delivered into cells by the method of an embodiment of the present invention: (A) is a fluorescence micrograph which shows expression of YFP proteins exhibiting yellow fluorescence; (B) is micrograph in the same position as in (B); (C) is fluorescence micrograph which shows expression of YFP proteins in a single cell; and (D) is a micrograph in the same position as in (C);

FIG. 8 shows material delivery efficiency and cell survival ratio when a genetic material was delivered by electroporation into cells respectively with the electroporation apparatus of an embodiment of the present invention and the commercialized electroporation apparatus: ‘Control’ is for the case that a DNA was introduced without electroporation; ‘Commercialized Apparatus’ is for the experimental result of electroporation which was obtained with the commercialized apparatus in the same condition as applied for the present invention; and ‘Present Invention’ is for the experimental result of electroporation which was obtained with the apparatus and method of the present invention.

### DETAILED DESCRIPTION OF INVENTION

Charging of a droplet by a direct contact with an electrode (hereinafter, it is sometimes called as ‘contact charging’) is a phenomenon that when a conductive liquid droplet contacts with an electrode, it is charged in the same polarity as that of the electrode by receiving an electrical charge from the surface of an electrode. The droplet charged as such moves to the opposite electrode by electrophoresis due to its electrical repulsion. When such contact charging and electrophoresis are applied, it is possible to drive individual droplet with electricity. Therefore, each droplet containing cells and a material to be delivered into the cells can be respectively controlled, and electroporation can be performed to droplets, by which the material can be delivered into the cells. By applying such phenomenon, the present invention provides an electroporation device which can be used for delivering a material conveniently and effectively to cells, an electroporation apparatus comprising the same, and a method for electroporation.

Therefore, a first aspect of the present invention relates to an electroporation device comprising: a droplet driving electrode for forming a mixed droplet comprising a first droplet containing cells and a second droplet containing a material to be delivered into the cells; and an electroporation electrode for performing electroporation on cells in the mixed droplet. The droplet driving electrode respectively contacts and charges the first droplet and the second droplet, so as to make each droplet moved by electrophoresis, by which the mixed droplet is generated.

The electroporation device of the present invention may comprise a plurality of droplet driving electrodes. In this case, the plurality of droplet driving electrodes may be arranged vertically on the internal surface of the electroporation device. Each of the plurality of droplet driving electrodes may independently apply an electrical voltage. Herein, at least one of the droplet driving electrodes may apply high voltage of electricity, and thus, it may function as an electroporation electrode.

The electroporation electrode can apply voltage to a mixed droplet that a first droplet and a second droplet are combined, by which an electroporation is carried out to the cells in the mixed droplet. The electroporation electrode and the droplet driving electrode may face each other in a way that the electroporation electrode is placed opposed and spaced to the internal surface of electroporation device where the droplet driving electrode is arranged. In this case, the space between the electroporation electrode and the droplet driving electrode may be adjusted.

In one embodiment of the electroporation device of the invention, both the droplet driving electrode and the electroporation electrode may be arranged vertically on the internal surface of the electroporation device.

When the electroporation device of the invention is used, one end and the other end of a mixed droplet, which contains cells and a material to be delivered into the cells, are connected respectively to the droplet driving electrode and the electroporation electrode, and a voltage with an electrical current penetrating the mixed droplet is applied by the droplet driving electrode and the electroporation electrode, by which an electroporation is performed.

In another embodiment of the invention, the electroporation electrode may be a needle type having a hollow inside, and thus, it can be connected to a syringe introduced from outside of the electroporation device thereto. In this case, the electroporation electrode of needle type can be connected to a syringe so as to collect cells that a material is delivered by electroporation.

A second aspect of the present invention relates to an electroporation apparatus comprising: an electroporation device according to the first aspect of the invention; a first power supply for applying voltage to the droplet driving electrode; and a second power supply for applying voltage to the electroporation electrode.

In performing electroporation using the electroporation apparatus of the invention, if necessary, the polarity and the voltage of the droplet driving electrode may be controlled with a controller which controls power of the first power supply. In addition, if desired, the electroporation apparatus may be controlled with a computer system that an algorithm and process for electrophoresis and electroporation of droplets are stored, by which components of the apparatus may be controlled. However, neither the controller nor the computer system is an essential component of the apparatus of the invention.

A third aspect of the present invention relates to a method for electroporation comprising: introducing into an electroporation device a first droplet containing cells and a second droplet containing a material to be delivered into the cells; combining the first droplet and the second droplet so as to generate a mixed droplet; and performing an electroporation to the cells in the mixed droplet.

In the step of combining the first droplet and the second droplet, the first droplet and the second droplet are charged by contacting with different droplet driving electrodes each other, and then, the charged first and second droplets are combined into a mixed droplet.

In the step of performing an electroporation, one end and the other end of a mixed droplet are connected respectively to the droplet driving electrode and the electroporation electrode, and a voltage with an electrical current penetrating the mixed droplet is applied by the droplet driving electrode and the electroporation electrode, by which an electroporation is performed. Through this, a material is delivered into cells.

One embodiment of the third aspect of the invention further comprises a step of collecting the mixed droplet with a syringe to be introduced from outside of the electroporation device through hollow of electroporation electrode in needle shape, after the step of performing electroporation.

In another embodiment of the third aspect, it is preferred that the electroporation method of the invention is performed in an electroporation device which is filled with hydrophobic insulting oil. In this case, all of the first droplet containing cells, the second droplet containing a material to be delivered into the cells, and the mixed droplet exist in oil phase, and thus, it is possible to deal with the cells and the material to be delivered into the cells without any risk of contamination.

In another embodiment of the invention, the method for electroporation may be carried out by introducing to the electroporation device according to the present invention a droplet containing both cells and a material to be delivered into the cells; and performing an electroporation to the cells in the droplet. In this case, the method of the invention comprises a step of introducing a droplet containing both cells and a material to be delivered into the cells to the electroporation device according to the present invention; and performing an electroporation to the cells in the droplet. The step of performing an electroporation is carried out in the same manner as described above.

A fourth aspect of the present invention relates to a method for delivering a material into cells comprising, performing electroporation of a first droplet containing cells and a second droplet containing a material to be delivered into the cells using an electroporation device according to the first aspect of the present invention, so as to deliver the material into the cells.

In the present invention, the material to be delivered into cells is any material which can be contained in a droplet and is desired to be delivered into the cells. Example of such material may be selected from the group consisting of a genetic material, a pharmaceutical, a fluorescence material and any combinations thereof, but not limited thereto.

In addition, the droplet containing cells and the material to be delivered into the cells may be any aqueous droplets or any liquid droplets which are immiscible with oil. Example of a media for the droplets may include water, polyethylene glycol (PEG), a buffer, an ionic liquid and the like, but not limited thereto.

Hereinafter, further embodiments of the present invention will be described in more detail with reference to the attached drawings.

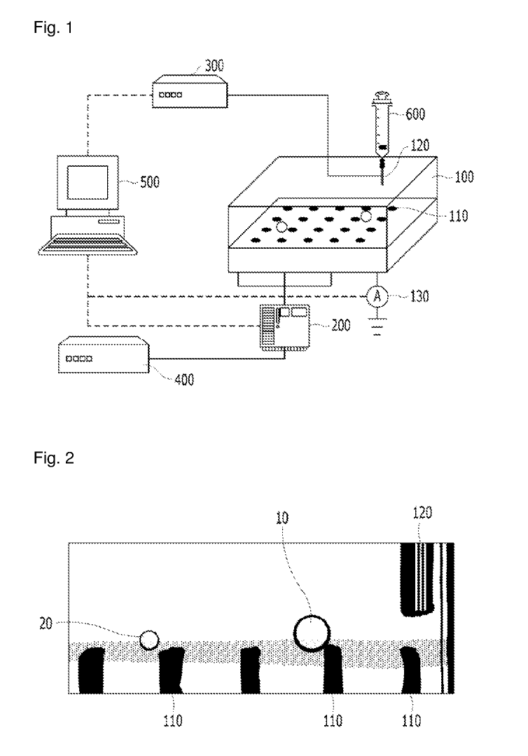

FIG. 1 shows main components of the electroporation apparatus of one embodiment of the present invention. The electroporation apparatus of the invention may comprise an electroporation device (100), a first power supply (200) and a second power supply (300) as illustrated in FIG. 1. Therefore, the electroporation device (100) as illustrated in FIG. 1 is one embodiment of the electroporation device of the present invention. The electroporation device of the first aspect of the present invention will be described in detail below with reference to FIGS. 2 to 4.

Electroporation Device

In one embodiment, the electroporation device (100) of the present invention comprises a droplet driving electrode (110) and an electroporation electrode (120) as illustrated in FIG. 1.

The droplet driving electrode (110) directly contacts with a first droplet (10) containing cells and a second droplet (20) containing a material, for example, a genetic material, which are introduced from outside of the electroporation device by a pipet or syringe, thereby charging the first droplet (10) and the second droplet (20). The droplets charged as such are respectively moved by electrophoresis and combined, thereby forming a mixed droplet (30).

In another embodiment, a droplet containing cells and a droplet containing a material to be delivered into the cells may be combined into a single droplet before it is introduced into an electroporation device.

As shown in FIG. 1, it is preferred that a plurality of the droplet driving electrode (110) may be equipped and arranged vertically on the internal surface of the electroporation device (100).

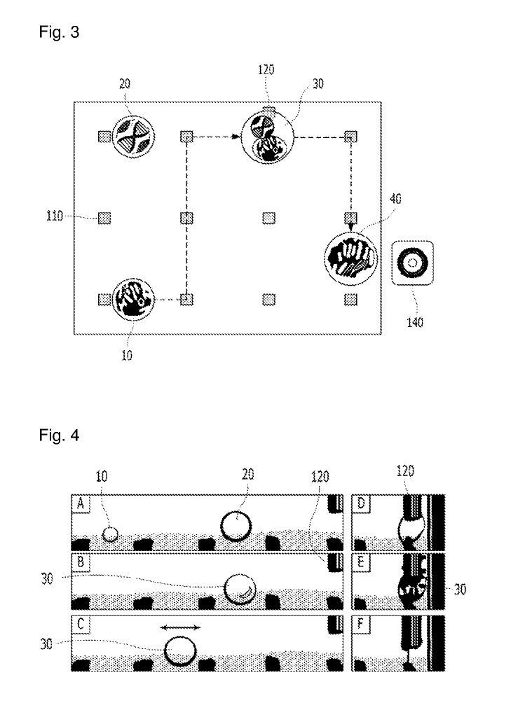

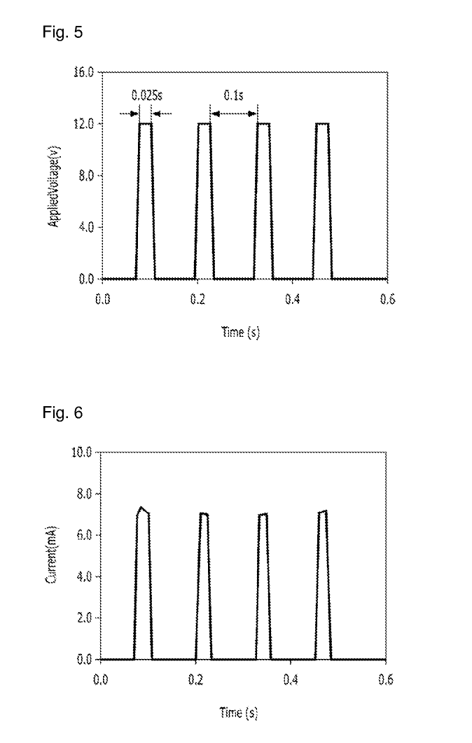

As shown in FIG. 3, the mixed droplet (30) which is formed by the droplet driving electrode (110) is transferred by electrophoresis to the electroporation electrode (120) for electroporation.

In order to minimize influence of gravity on the droplets and friction between the droplets and the internal surface of the device, it is preferred that inside of the electroporation device (100) is filled with hydrophobic insulting oil. In this case, it is preferred to use oil of which viscosity is low, so as to minimize any resistance to be induced during the movement of the droplets. For example, any oil, of which viscosity is from half to hundreds times compared with water, may be used, and it is not limited to any specific kind if it is consistent with the object of the invention. If oil is filled in the electroporation device (100), contact portion of cells with the solid surface of the device is minimized, by which any contamination of cells can be prevented.

As depicted in FIGS. 2 and 4, an electroporation electrode (120) may be placed opposed and spaced to the internal surface of an electroporation device (100) where the droplet driving electrode (110) is arranged. When a mixed droplet (30) arrives at a position between the droplet driving electrode (110) and the electroporation electrode (120), a voltage required for electroporation is applied to the electroporation electrode (120) from a second power supply (300), by which electroporation is performed to cells in the mixed droplet (30). The space between the droplet driving electrode (110) and the electroporation electrode (120) may be adjusted in accordance with the size of droplets to be introduced. The space is preferably adjusted in the level that both electrodes can be connected with each other through the mixed droplet (30).

The process of electroporation to cells in the mixed droplet (30) can be observed by detecting current change during the electroporation with an ammeter (130) which is connected to the droplet driving electrode (110).

After electroporation with current penetrating the mixed droplet (30), the mixed droplet (30) are collected and cultivated for the use of further analysis, etc. The electroporation electrode (120) may be made in needle shape having hollow, and thus, one end of the electroporation electrode (120) can be connected with the syringe (600) introduced from outside of the electroporation device (100), by which the mixed droplet may be collected with oil by the syringe (600) through the hollow after completion of electroporation.

FIG. 3 shows the process of performing electroporation and delivering a material into cells inside the electroporation device in which its components were integrated and automated according to an embodiment of the present invention. As illustrated in FIG. 3, an electroporation electrode (120) may be placed on the internal surface of the electroporation device (100), so as to make the integration of the device be easy. In addition, it is preferable to make it possible to observe the level of material delivery into cells through observation portion (140), and also to perform electroporation repeatedly if additional electroporation is desired. In this case, electroporation may be performed only with direct chagin of droplets and electrophoresis, in addition to the way that a voltage by current penetrating through the droplets is applied, by which one end and the other end of the droplets are respectively connected with the electroporation electrode (120) and the droplet driving electrode (110).

In another embodiment, the electroporation device of the invention may not have any electroporation electrode (120). In this case, at least one of the droplet driving electrodes (110), which are arranged on the internal surface of the device, may be made such that it can apply high voltage, and thus, electroporation can be achieved only with electrophoresis.

Electroporation Apparatus

As depicted in FIG. 1, the electroporation apparatus of the invention comprises an electroporation device (100), a first power supply (200) and a second power supply (300). The first power supply (200) applies a voltage to a droplet driving electrode (110), and the second power supply (300) applies a voltage to an electroporation electrode (120).

If desired, the electroporation apparatus of the invention may further comprise a controller (400). The controller (400) controls the polarity of the droplet driving electrode (110), and also controls the power of the first power supply (200) so as to adjust the applied voltage of the droplet driving electrode (110).

If a plurality of droplet driving electrodes (110) is equipped, the controller (400) and the first power supply (200) are made to control each droplet driving electrode (110) such that each droplet driving electrode (110) can independently apply a voltage.

If desired, the electroporation apparatus may be controlled with a computer system (500), in which an algorithm and process for electrophoresis and electroporation of droplets are stored, by which components of the apparatus may be controlled. However, neither the controller nor the computer system is an essential component of the apparatus of the invention.

Method for Electroporation and Method for Delivery of Materials

In the electroporation method of the present invention, a first droplet containing cells (10) and a second droplet containing a material to be delivered to the cells (20) are introduced into the electroporation device (100), the first droplet (10) and the second droplet (20) are charged in contact with the droplet driving electrode (110) included in the electroporation device (100) so as to form a mixed droplet (30), and then electroporation is performed to the mixed droplet (30). In such a way, a material is delivered into cells in the mixed droplet (30) in accordance with the present invention.

Herein, one end and the other end of the mixed droplet (30) are connected respectively with the droplet driving electrode (110) and the electroporation electrode (120), the electroporation electrode (120) applies a voltage penetrating the mixed droplet (30), by which electroporation is performed to the mixed droplet (30).

After electroporation, the mixed droplet (30) can be collected with a syringe (600) connected from outside of the device to an electroporation electrode (120) which is in needle shape having hollow and can be connected with the syringe (600).

FIG. 4 demonstrates a process of a droplet driving, a droplet combination and electroporation according to an embodiment of the present invention.

As depicted in FIGS. 4A and 4B, a first droplet containing cells (10) and a second droplet containing a material to be delivered to the cells (20) are introduced into the electroporation device (100) with a pipet or syringe, and then, the polarity of the droplet driving electrode (110) on the internal surface of the electroporation device (100) is controlled, by which the first droplet (10) and the second droplet (20) are moved and combined by electrophoresis, so as to form a mixed droplet (30).

As depicted in FIG. 4C, cells and the material to be delivered thereto are well mixed in the mixed droplet (30), and then the polarity of the droplet driving electrode (110) is controlled so as to make the mixed droplet (30) transferred to the electroporation electrode (120) by electrophoresis.

As depicted in FIGS. 4D and 4E, electroporation to the transferred mixed droplet is performed by current penetrating the mixed droplet (30), and then, the mixed droplet is collected with oil by a syringe (600) which is connected to one end of the electroporation electrode (120) in needle shape.

Even though the present invention uses a droplet, of which volume is below a few microliters, cells from hundreds of thousands to several millions can be included in the droplet. Since the droplet size used in the present invention is small, voltage below several tends of volts and current below several tends of amperes can be applied for electroporation. Therefore, in the present invention, it is not necessary to use any expensive apparatus which can apply high voltage and high current, which results in cost reduction for manufacturing an electroporation device and apparatus.

It is also possible to deliver a material to a large number of cells with a small-sized droplet by electroporation, and thus, an experiment can be carried out even with a small amount of cells and materials to be delivered thereto in high concentration, by which higher material delivery efficiency can be achieved compared with a commercialized apparatus.

It is also possible to deal with a large amount such as from hundreds of thousands to millions of cells at once. Therefore, limitations of the prior art microfluidic electroporation device, of which productivity is lower than that of the commercialized apparatus, can be overcome.

### EXAMPLES

Hereinafter, the processes and results obtained from electroporation with an apparatus and method of the present invention are described in detail.

Cells of Chlamydomonas reinhardtii which is unicellular micro green algae having cell wall were used. The delivery of any material to those cells has been known to be difficult because of their thick cell wall. As a genetic material, plasmid DNA which can generate yellow fluorescence protein (YFP) was used.

Electroporation was performed with 200,000 of cells of micro green algae and 14,000,000,000 of DNA in about 1 μl (diameter of 1 mm) of cell culture (Tris-Acetate-Phosphate, TAP medium). The space between the droplet driving electrode and the electroporation electrode was kept at 1 mm. Silicone oil was filled in the device. Electric field for electroporation was set to 480 V/cm both for the commercialized electroporation apparatus (192 V/4 mm space) and the electroporation apparatus of the present invention (48 V/1 mm space). Voltage pulses were applied eight times with 50 ms and intervals between pulses were set to 100 ms equally.

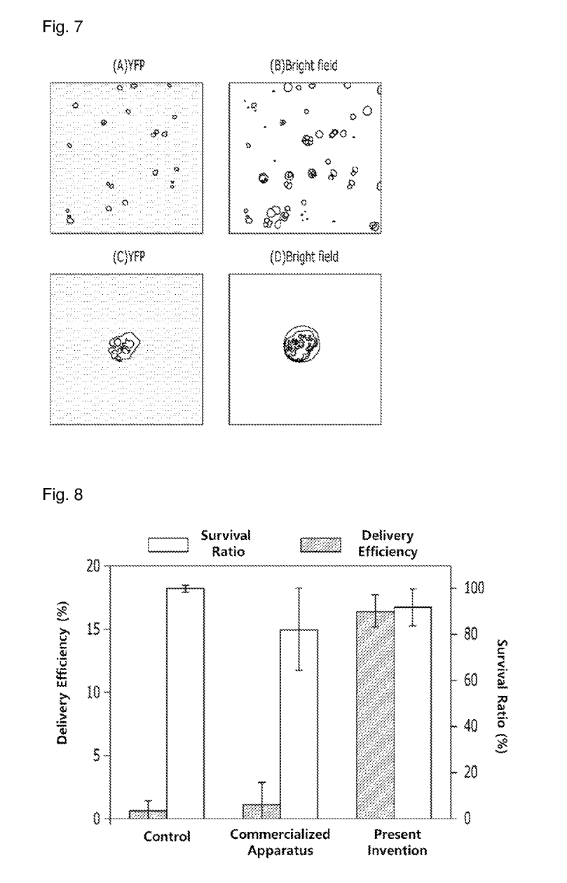

Experimental results of genetic material delivery efficiencies to actual cells and cell survival ratios were presented in FIGS. 7 and 8.

FIG. 7 shows photos identifying the existence of fluorescence protein which was formed in cells after delivering a gene, which express yellow fluorescence protein, into cells, in accordance with the process of electroporation as depicted in FIG. 4.

FIGS. 7(A) and 7(C) are fluorescence images in darkroom, and FIGS. 7(B) and 7(D) are images of the outlines of cells which were taken at the same position under the light. FIGS. 7(A) and 7(C) show that a genetic material, which can generate a protein exhibiting fluorescence, was successfully delivered into cells, and therefore, the protein was successfully expressed. Further, FIGS. 7(B) and 7(D) clearly show that the protein was properly expressed in cells by the genetic material which was delivered into the cells.

FIG. 8 shows material delivery efficiency and cell survival ratio when a genetic material was delivered by electroporation into cells in accordance with the process as shown in FIG. 4, and those obtained with the commercialized electroporation apparatus (Bio-Rad, Gene Pulser Xcell). In FIG. 8, Control’ is for the case that a DNA was introduced without electroporation; ‘Commercialized Apparatus’ is for experimental result of electroporation which was obtained with the commercialized apparatus in the same condition as applied for the present invention; and ‘Present Invention’ is for experimental result of electroporation which was obtained with the apparatus and method of the present invention. Electric field for electroporation was set to 480 V/cm both for the commercialized electroporation apparatus and that of the present invention. As shown in FIG. 8, the cell survival ratio is relatively low (82%) due to high voltage (192 V) and high current (2.8 A) and the genetic material delivery efficiency is only 1.1% for the commercialized apparatus. Whereas in the present invention, the same electric field could be applied with low voltage (48 V) and low current (0.02A) since the apparatus of the present invention is small, which results in relatively high cell survival ratio (92%) with 16.5% of genetic material delivery ratio.

The examples and embodiments presented in the specification and drawings attached thereto are only for illustrating and exemplifying parts of the technical concept of the present invention. Therefore, it is obvious that the scope of the present invention is not limited to those examples and embodiments, which were presented for illustrating not for limiting the technical concept of the present invention.

Therefore, it should be understood that any modifications or embodiments which can be easily inferred by a person of ordinary skill in the art within the scope of the technical concept included in the specification and the drawings attached thereto are included in the scope of the present invention.

### INDUSTRIAL APPLICABILITY

The electroporation device, the apparatus comprising the same, and electroporation according to the present invention can be applied for any device to deliver a material into cells, such as a small cell incubator, a cell engineering device, and the like. Therefore, it is applicable in the field of chemistry, biological science, medicine, pharmacy, and the like, which uses micro-fluid dynamics.

DESCRIPTION OF NUMERALS OF DRAWINGS

| | |
|---|---|
| 10: first droplet | 20: second droplet |
| 30: mixed droplet | 100: electroporation device |
| 110: droplet driving electrode | 120: electroporation electrode |
| 130: ammeter | 140: observation portion |
| 200: first power supply | 300: second power supply |
| 400: controller | 500: computer system for control |
| 600: syringe | |

1. An electroporation device comprising:
a droplet driving electrode generating a mixed droplet comprising a first droplet containing cells and a second droplet containing a material to be delivered to the cells; and
an electroporation electrode for performing electroporation to the cells in the mixed droplet.

2. The electroporation device according to claim 1, wherein the droplet driving electrode contacts with and charges the first droplet and the second droplet, respectively, so as to make the first droplet and the second droplet moved by electrophoresis to form the mixed droplet.

3. The electroporation device according to claim 1, comprising a plurality of droplet driving electrodes which are arranged vertically on the internal surface of the electroporation device.

4. The electroporation device according to claim 3, wherein each of the plurality of droplet driving electrodes can independently apply a voltage.

5. The electroporation device according to claim 4, wherein at least one of the plurality of droplet driving electrodes can apply a high voltage for electroporation and it functions as the electroporation electrode.

6. The electroporation device according to claim 1, wherein the electroporation electrode applies a voltage to the mixed droplet comprising the first droplet and the second droplet, so as to perform electroporation on the cells in the mixed droplet carried out.

7. The electroporation device according to claim 3, wherein the electroporation electrode and the droplet driving electrode face each other in a way that the electroporation electrode is placed opposed and spaced to the internal surface of electroporation device where the droplet driving electrode is arranged.

8. The electroporation device according to claim 7, wherein the space between the droplet driving electrode and the electroporation electrode can be adjusted.

9. The electroporation device according to claim 7, wherein the one end and the other end of the mixed droplet are connected respectively with the droplet driving electrode and the electroporation electrode, and a voltage is applied by a current penetrating the mixed droplet through the droplet driving electrode and electroporation electrode, by which the electroporation is performed on the cells in the mixed droplet.

10. The electroporation device according to claim 1, wherein both the droplet driving electrode and the electroporation electrode are arranged vertically on the internal surface of the electroporation device.

11. (canceled)

12. The electroporation device according to claim 1, wherein the electroporation electrode is in needle shape and can be connected to a syringe which is introduced from outside of the electroporation device.

13. The electroporation device according to claim 1, wherein the material to be delivered into cells is selected from the group consisting of a genetic material, a pharmaceutical, a fluorescence material and any combinations thereof.

14. (canceled)

15. A method for electroporation comprising:
a step of droplet introduction comprising, introducing to an electroporation device a first droplet containing cells and a second droplet containing a material to be delivered to the cells;
a step of a mixed droplet generation comprising, combining the first droplet and the second droplet so as to generate a mixed droplet; and
a step of electroporation comprising, performing electroporation to the cells in the mixed droplet.

16. The method for electroporation according to claim 15, wherein in the step of mixed droplet generation, the first droplet and the second droplet are respectively contacted with different droplet driving electrodes and charged thereby, and then the first and second droplets are moved by electrophoresis, so as to be combined with each other to form the mixed droplet.

17. The method for electroporation according to claim 15, wherein in the step of electroporation, one end and the other end of the mixed droplet are connected respectively with the droplet driving electrode and the electroporation electrode, and a voltage is applied by a current penetrating the mixed droplet through the droplet driving electrode and electroporation electrode, by which the electroporation is performed on the cells in the mixed droplet.

18. The method for electroporation according to claim 15, further comprising a step of collecting the mixed droplet with a syringe through hollow of an electroporation electrode in needle shape having the hollow after the step of electroporation.

19. The method for electroporation according to claim 16claim 15, wherein the electroporation is performed in hydrophobic insulating oil which is filled in the electroporation device.

20. The method for electroporation according to claim 15, wherein the material to be delivered into cells is selected from the group consisting of a genetic material, a pharmaceutical, a fluorescence material and any combinations thereof.

21. A method for electroporation comprising:
a step of introducing a droplet containing cells and a material to be delivered to the cells into an electroporation device according to claim 1; and
a step of performing electroporation to the cells in the droplet.

22. The method for electroporation according to claim 21, wherein the material to be delivered into cells is selected from the group consisting of a genetic material, a pharmaceutical, a fluorescence material and any combinations thereof.

23.-24. (canceled)
